(19) United States
(12) Patent Application Publication
(10) Pub. No.: US 2026/0130776 A1
(43) Pub. Date: May 14, 2026

(54) IMPLANTABLE DEVICE WITH ENHANCED DRUG DELIVERY AREA

(71) Applicant: Envision Scientific Private Limited, Gujarat (IN)

(72) Inventors: Manish Indulal Doshi, Surat (IN); Prakash Nanjibhai Sojitra, Surat (IN); Parth Manish Doshi, Surat (IN); Dinesh Shah, Troy, MI (US)

(21) Appl. No.: 19/445,463

(22) Filed: Jan. 9, 2026

Related U.S. Application Data
(63) Continuation-in-part of application No. 16/636,891, filed on Feb. 5, 2020, now abandoned.

(51) Int. Cl. A61F 2/90; A61F 2/958

(52) U.S. Cl. CPC A61F 2/90; A61F 2/958; A61F 2210/0076; A61F 2250/0067

(57) ABSTRACT

An apparatus for delivering a drug to a vascular body lumen includes a pre-crimped stent configured to be disposed within a vascular body lumen and a coating. The stent is mounted on a balloon and includes a mesh-like configuration having multiple interconnected space regions defined by multiple strut components. Each of the strut components include an abluminal surface, a luminal surface, and a side surface. The balloon includes multiple portions exposed through the interconnected space regions. The coating bridges across the interconnected space regions of the stent and is configured to deform and extend in response to expansion of the balloon to form a continuous, homogenous cylindrical film. Expansion of the balloon within the vascular body lumen causes the homogenous cylindrical film to cover a continuous, circumferential area of the vascular body lumen.

### CROSS-REFERENCE TO RELATED APPLICATIONS

This application claims the benefit of priority of U.S. patent application Ser. No. 16/636,891, filed on Feb. 5, 2020, the contents of which are relied upon and incorporated herein by reference in their entirety

### TECHNICAL FIELD

This disclosure generally relates to a drug delivery medical apparatus. More specifically, the disclosure relates to a homogenously coated implantable device with enhanced drug delivery area providing maximum coverage target lesions within a vascular lumen.

### BACKGROUND

Significant advances have been made in the treatment of coronary artery disease (CAD), particularly through the transition from bare metal stents to drug-eluting stents (DES). Drug-eluting stents have demonstrated a substantially reduced rate of restenosis compared to bare metal stents and are widely used to reopen occluded arteries, restore blood flow, and reduce the likelihood of re-narrowing following implantation. Despite these advantages, existing DES technologies continue to exhibit important limitations, leaving substantial opportunity for improvement. These limitations are especially pronounced in certain clinical indications, including diabetic patients, acute myocardial infarction patients, bifurcation lesions, and chronic total occlusions (CTO). For example, patients with diabetes, including those with below-the-knee vascular disease, frequently experience suboptimal outcomes with currently available DES systems.

Conventional drug-eluting stents are coated primarily on the metallic surface of the stent struts. As a result, drug delivery is largely confined to regions of direct contact between the stent struts and the vessel wall. When implanted, these contact regions typically correspond to only approximately 12-20% of the total luminal surface area, leaving a majority of the vessel wall untreated and deficient in therapeutic drug exposure.

In addition, lipophilicity plays a critical role in governing in-tissue drug diffusion and drug residence time, both of which directly affect bioavailability. In current CAD treatments, DES devices predominantly deliver limus-based drugs. Following implantation, these drugs are released mainly from the contact regions of the stent struts, which again represent only a limited fraction of the vessel lumen surface area. Consequently, diffusion of the drug into the arterial wall is restricted, resulting in inadequate treatment of large portions of diseased tissue. These untreated regions contribute to restenosis and re-blockage, with the incidence and severity varying among patients based on individual physiological conditions. Diabetic patients in particular often exhibit diffuse, proliferative, and continuous disease characterized by reduced lumen diameter and extended lesion length, further complicating effective drug delivery and exacerbating these limitations.

From a bioavailability perspective, the limitation of coating only the stent surface inherently restricts the total amount of drug delivered to the vessel wall. Because drug release is confined to the area covered by the stent struts, both drug exposure and spatial distribution are limited. Although limus-based drugs used in existing DES systems have demonstrated clinical safety, they are associated with relatively poor bioavailability, rapid washout from the vessel wall, and limited in-tissue diffusion. These factors contribute to the persistence of untreated regions within the vessel and increase the likelihood of disease recurrence.

Accordingly, there remains a need in the art for an improved drug delivery device capable of providing enhanced, area-based drug delivery to a vascular lumen or artery. Such a device should be effective irrespective of drug lipophilicity or inherent bioavailability characteristics and should deliver therapeutic agents uniformly to the entire diseased region of the vessel wall, thereby reducing restenosis and inhibiting uncontrolled cellular proliferation.

### BRIEF SUMMARY

According to one aspect of the present disclosure, an apparatus for delivering a drug to a vascular body lumen includes a pre-crimped stent and a coating. The pre-crimped stent is mounted on a balloon and configured to be disposed within a vascular body lumen defined by a vascular body wall. The pre-crimped stent includes a mesh-like configuration having multiple interconnected space regions defined by multiple strut components. Each of the strut components include an abluminal surface, a luminal surface, and a side surface. The balloon includes multiple exposed portions exposed through the interconnected space regions and multiple adjacent sections beneath the luminal surface.

The coating contacts the abluminal surface and the side surface of each of the strut components as well as the exposed portions of the balloon and the adjacent sections of the balloon lack coating. The coating bridges across the interconnected space regions of the pre-crimped stent. Further, the coating is configured to deform and extend in response to expansion of the balloon to form a continuous, homogenous cylindrical film circumscribing the abluminal surface. Formation of the homogenous cylindrical film is driven by expansion of the balloon. The homogenous cylindrical film includes a polymer matrix including at least one drug and one or more polymers from a poly-L-lactide family. Upon expansion of the balloon within the vascular body lumen, the homogenous cylindrical film covers a continuous, circumferential area of the vascular body wall.

In some embodiments, the coating further contacts a proximal end and a distal end of the balloon such that the homogenous cylindrical film further substantially circumscribes the proximal end and the distal end. In some embodiments, deformation of the coating in response to expansion of the balloon includes tensile stretching of the coating across the interconnected space regions.

In some embodiments, upon expansion of the balloon within the vascular body lumen, the homogenous cylindrical film contacts a surface of the vascular body wall. In certain embodiments, the homogenous cylindrical film continuously coats the circumferential surface. In some embodiments, the homogenous cylindrical film remains continuously in contact with the circumferential surface upon deflation of the balloon.

In some embodiments, the coating includes a polymer having an elasticity and a plasticity sufficient to deform without cracking in response to expansion of the balloon. The polymer may further include a molecular weight sufficient to permit tensile deformation in response to expansion of the balloon.

In some embodiments, the coating extends longitudinally onto the distal end and the proximal end of the balloon by a distance between 0.5 mm and 1.0 mm beyond a length of the pre-crimped stent. In some embodiments, the luminal surface of each of the plurality of strut components and the adjacent sections of the balloon beneath the luminal surface remain devoid of the coating after formation of the homogenous cylindrical film.

According to another aspect of the present disclosure, a system for delivering a drug to a vascular body lumen includes a balloon catheter, a pre-crimped stent, and a coating. The balloon catheter includes an expandable balloon and is configured to be disposed within a vascular body lumen. A pre-crimped stent is mounted on the balloon and includes multiple strut components defining a plurality of interconnected space regions.

The coating is disposed on an outer surface of the pre-crimped stent and on portions of the balloon exposed through the interconnected space regions. The coating bridges across the interconnected space regions of the pre-crimped stent. The coating is configured to deform and extend in response to expansion of the balloon to form a continuous, homogenous cylindrical film circumscribing the pre-crimped stent. Formation of the homogenous cylindrical film is driven by expansion of the balloon, and the homogenous cylindrical film circumferentially contacts an inner wall of the vascular body lumen in response to such expansion.

In some embodiments, wherein the pre-crimped stent includes multiple strut components, each of the plurality of strut components comprising an abluminal surface, a luminal surface, and a side surface, and wherein the outer surface of the pre-crimped stent corresponds to the abluminal surface and the side surface of each of the plurality of strut components. In some embodiments, balloon regions disposed beneath the luminal surface of each of the plurality of strut components remain devoid of the coating.

In certain embodiments, the deformation of the coating in response to expansion of the balloon includes tensile stretching of the coating across the interconnected space regions. The homogenous cylindrical film may thus be devoid of apertures corresponding to the interconnected space regions of the pre-crimped stent.

In some embodiments, the coating includes a polymer selected to have an elasticity and a plasticity sufficient to deform without cracking in response to expansion of the balloon. In certain embodiments, the polymer includes a molecular weight selected to permit tensile deformation in response to expansion of the balloon.

In some embodiments, the coating extends longitudinally onto the distal end and the proximal end of the balloon by a distance between 0.5 mm and 1.0 mm beyond a length of the pre-crimped stent. The coating may include a polymer matrix containing one or more drugs selected from the group consisting of anti-restenotic, anti-proliferative, anti-inflammatory, antithrombotic, immunosuppressive, and cytostatic agents.

In some embodiments, the homogenous cylindrical film is formed in vivo in response to the coating coming into contact with fluid within the vascular body lumen.

### BRIEF DESCRIPTION OF DRAWINGS

FIG. 1 is an end view of one example of an apparatus for delivering a drug to a vascular body lumen prior to balloon expansion, in accordance with some embodiments of the disclosure;

FIG. 2 is a cross-sectional view of one example of an apparatus of the resultant coating formation disposed within a vascular body, illustrating balloon expansion in accordance with some embodiments of the disclosure;

FIG. 3 is a cross-sectional of the apparatus of FIG. 2, illustrating one example of coating disposed on the inner surface of a vessel wall after removal of the balloon, in accordance with some embodiments of the disclosure;

FIG. 4A is a perspective view of one embodiment of the apparatus disposed within a vascular body with the balloon partially inflated in accordance with some embodiments of the disclosure;

FIG. 4B is a perspective view of the apparatus and vascular body of FIG. 4A, illustrating the balloon fully inflated in accordance with certain embodiments of the disclosure;

FIG. 5 is perspective view of one embodiment of the apparatus mounted on a representative balloon catheter, illustrating the coating bridging the interconnected space regions and extending over the ends of the balloon in accordance with some embodiments of the disclosure;

FIG. 6 is a graph of one example of results of an HPLC analysis of coating coverage of the inner surface of a vascular body over time following use of the apparatus within a vascular body in accordance with some embodiments of the disclosure; and

FIG. 7 is a graph of multiple example results of HPLC analyses of coating coverage of the inner surface of a vascular body over time following use of the apparatus utilizing a standard/control solution in accordance with some embodiments of the disclosure.

### DETAILED DESCRIPTION

Before describing the embodiments of the present disclosure in detail, it is noted that the embodiments described herein relate primarily to combinations of structural and functional components of an implantable drug delivery system comprising a stent and a balloon catheter. In particular, the disclosed embodiments relate to a coated, pre-crimped stent mounted on a balloon catheter, in which both the stent and selected portions of the balloon cooperate to deliver a therapeutic agent to a vascular body lumen. The description is presented to enable a person skilled in the art to make and use the disclosed system and is not intended to limit the scope of the claims to the specific embodiments described.

As used herein, the term “abluminal surface” refers to the surface of a stent, strut component, or implantable structure that is oriented away from the lumen of a vessel or body passage and toward the surrounding tissue, such as a vessel wall, after implantation. In the context of a vascular stent, the abluminal surface is the surface intended to contact or face the vessel wall when the stent is deployed.

The term “luminal surface” refers to the surface of a stent, strut component, or implantable structure that is oriented toward the lumen of a body passage and faces the flow path of blood or bodily fluid after implantation. In the context of a vascular stent, the luminal surface is the surface exposed to blood flow within the vessel after deployment.

As used herein, the term “side surface” refers to a surface of a stent strut component that extends between the abluminal surface and the luminal surface and is oriented substantially radially or laterally relative to the longitudinal axis of the stent. The side surface typically forms the thickness dimension of the strut and may face adjacent struts or interconnected space regions within the stent structure.

As used herein, the term “interconnected space regions” refers to open regions defined between adjacent strut components of the stent in a pre-crimped state. The area and geometry of the interconnected space regions are not fixed and increase as the stent expands radially in response to balloon expansion.

As used herein, deformation of the coating “in response to expansion of the balloon” refers to mechanical deformation caused by externally applied radial expansion forces of the balloon, including tensile stretching across the interconnected space regions, and does not include self-expansion, swelling, hydration-activated expansion, or shape-memory behavior of the coating.

Various embodiments of the disclosure provide an implantable drug delivery system configured to deliver a therapeutic agent circumferentially to a vascular body lumen. The system combines a permanent implant (a stent) with a temporary delivery structure (a balloon catheter), such that drug delivery is not limited solely to the contact regions of stent struts with the vessel wall.

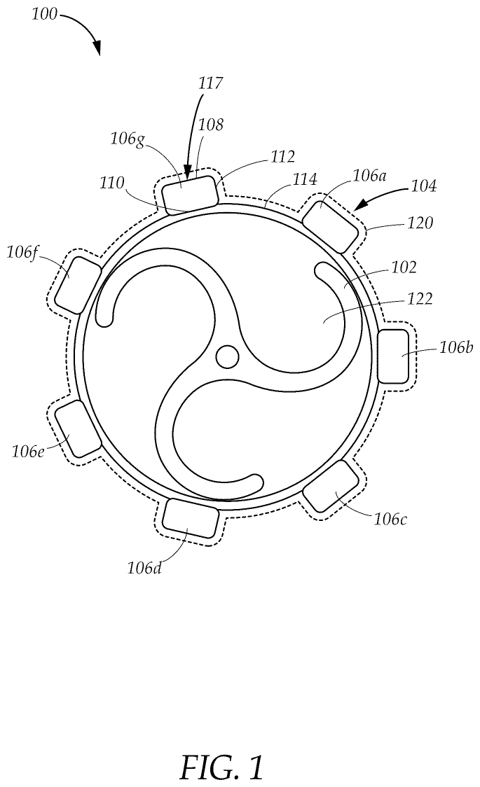

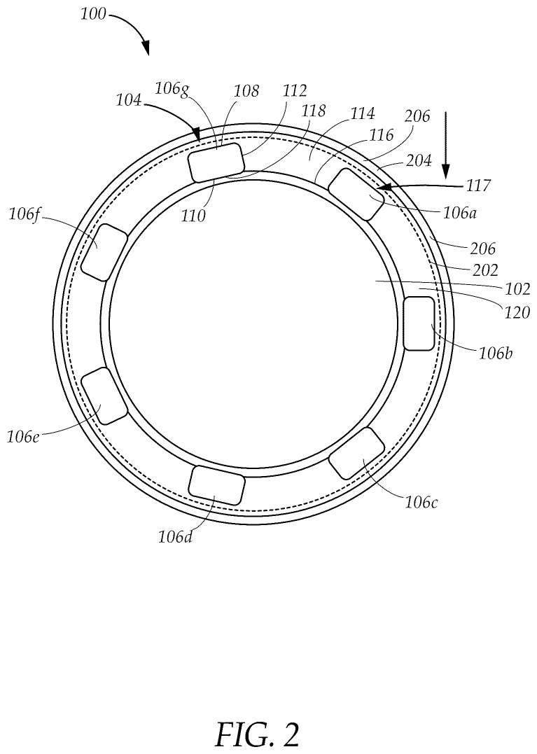

Referring now to FIGS. 1 and 2, in some embodiments, an apparatus 100 for delivering a drug to a vascular body lumen 122 may be configured to deliver one or more drugs to a treatment site in a coronary and/or peripheral vascular artery or vessel. In some embodiments, the apparatus 100 is a single consolidated drug delivery medical apparatus 100.

In some embodiments, the apparatus 100 includes a stent 104 mounted on an expandable balloon 102. In some embodiments, the stent 104 is pre-crimped. The stent 104 includes a mesh-like configuration having multiple interconnected space regions 114 defined by multiple strut components 106a-106g.

In the pre-crimped state, each interconnected space region 114 may include an area defined by the relative spacing and orientation of adjacent strut components 106a-106g. Upon expansion of the balloon 102, radial expansion of the stent 104 may cause the strut components 106a-106g to separate and reorient, thereby increasing the area of each interconnected space region 114.

For example, in a pre-crimped configuration suitable for delivery through a vascular lumen 122, an interconnected space region 114 may have a characteristic width in a range of about 50 μm to about 150 μm and a projected area in a range of about 0.01 mm² to about 0.05 mm². Upon expansion of the balloon 102 to a deployed configuration, the same interconnected space region 114 may expand to a characteristic width in a range of about 150 μm to about 400 μm and a projected area in a range of about 0.05 mm² to about 0.30 mm², depending on stent 104 design and target vessel diameter.

In some embodiments, each of the strut components 106a-106g includes an abluminal surface 108, a luminal surface 110, and a side surface 112. The balloon 102 includes multiple exposed portions 116 exposed through the interconnected space regions 114 and multiple adjacent sections 118 disposed beneath the luminal surfaces 110.

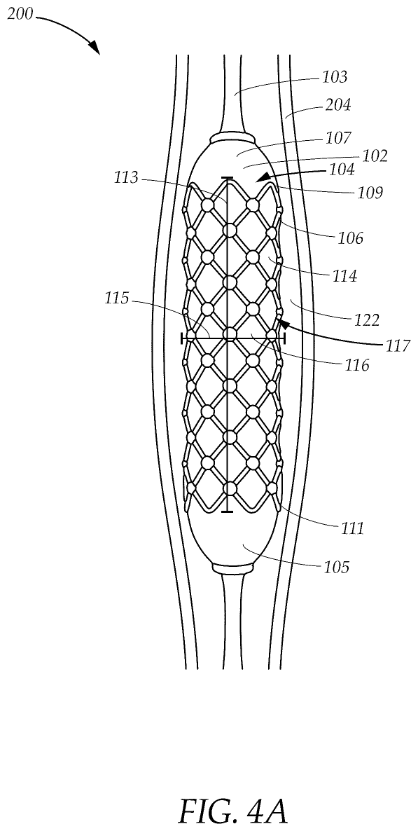

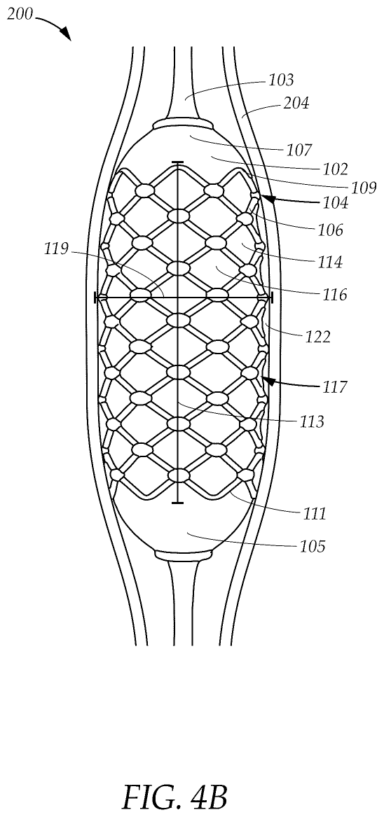

In some embodiments, such as the embodiment shown in FIG. 4A (while still referring to FIGS. 1 and 2), the stent 104 extends longitudinally between a first end 109 and a second end 111 and the interconnected space regions 114 are further defined by the first and the second ends 109, 111 to create a mesh like configuration. The crimping of the stent assembly 104 mounted on the balloon 102 may be performed by one or more suitable methods, including mechanisms utilizing stent crimping equipment and/or manual crimping methodologies.

A coating 120 is disposed on the stent 104 such that the coating 120 contacts the abluminal surface 108 and the side surface 111 of each of the strut components 106a-106g, as well as the exposed portions 116 of the balloon 102. The adjacent sections 118 of the balloon disposed beneath the luminal surfaces 110 of the strut components 106a-106g lack coating 120.

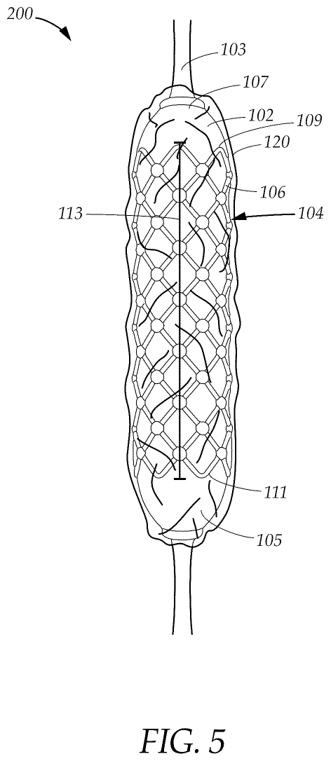

In some embodiments, the coating 120 bridges across the interconnected space regions 114 of the pre-crimped stent 104. The coating 120 may be further configured to deform and/or extend in response to expansion of the balloon 102 to form a continuous, homogenous cylindrical film 202 circumscribing the abluminal surface, as shown in FIG. 5. Formation of the homogenous cylindrical film 202 may be driven by expansion of the balloon 102.

Referring now to FIG. 5, while still referring to FIGS. 1 and 2, the homogenous cylindrical film 202 may include a polymer matrix including at least one drug and one or more polymers from a poly-L-lactide family. Upon expansion of the balloon 102 within a vascular body lumen 122, the homogenous cylindrical film 202 is configured to entirely cover a continuous, circumferential area of the vascular body wall 204.

In some embodiments, the coating 120 covers an outer surface 117 of the stent 104, including an abluminal surface 108 of each of the plurality of strut components 106a-106g and exposed regions 116 of the balloon 102 assembly radially extending and exposed through the plurality of interconnected space regions 114. In some embodiments, the coating 120 further covers a distal end 105 and/or a proximal end 107 of the balloon 102. The distal and proximal ends 105, 107 of the balloon 102 may be exposed beyond the first end 109 and the second end 28, respectively, of the pre-crimped stent 104. In some embodiments, the coating 120 may substantially circumscribe each the distal end 105 and/or the proximal end 107. In other embodiments, the coating 120 may extend along the distal end 105 and/or the proximal end 107 of the balloon 102 for a distance between about 0.5 mm and about 1.0 mm beyond a length 113 of the pre-crimped stent 104.

In some embodiments, the coating 120 includes an organic solvent soluble matrix of one or more drugs and one or more polymers. The drugs may be selected from a group including, but not limited to, an anti-restenotic agent, an anti-proliferative agent, an anti-inflammatory agent, an antithrombotic agent, and an antioxidant an immunosuppressive agent, a cytostatic agent and a cytotoxic agent. In certain embodiments, the one or more drugs are selected from a group including, but not limited to, sirolimus, tacrolimus, paclitaxel, beta-estadiol, rapamycin, everolimus, ethylrapamycin, zotarolimus, ABT-578, Biolimus A9 and analogs of rapamycin mitomycin, myomycine, novolimus, permirolast potassium, alpha-interferon, bioactive RGD and salts, esters or analogues thereof.

In still other embodiments, the drug includes, but is not limited to, one or more of sirolimus, tacrolimus, paclitaxel, heparin, beta-estradiol, rapamycin, everolimus, ethylrapamycin, zotarolimus, ABT-578, Biolimus A9, docetaxel and mitomycin.

In some embodiments, the one or more polymers are selected from a group including, but not limited to, a homopolymer; a co-polymer of glycolide and lactide; a co-polymer of trimethylene carbonate; e-caprolactone and polydiaxanone; Poly Glycolic Acid (PGA); Poly(Lactic-co-Glycolic Acid) (PLGA); Poly(Ethylene Glycol) (PEG); Polyglactin; Polyglyconate; Polydiaxanone; Polyglecaprone; Polyglycolide; Polylactide; Polyhydroxybutyrate; Poly(Glycolide-E-Caprolactone); Poly(Glycolide Trimethylene Carbonate); Poly(L-lactic Acide-L-lysine) copolymer; Tyrosine-based polyarylates; Polyiminocarbonates; Polycarbonates; Poly(D;L-lactide-Urethane); Poly(esteramide); Poly-P-Dioxanone; hyaluronic acid; chitin; chitosan; Poly-L-Glutamic Acid; Poly-L-Lysine; Polyphosphazene; Poly[bis(carboxylatophenoxy)phosphazene] and combinations thereof.

In one embodiment, the coating 120 includes a bio-degradable polymer matrix of Poly-L Lactide family in addition to the one or more drugs. A molecular weight of one or more of the polymers may be altered to achieve increased elasticity and plasticity limits. For example, in certain embodiments, the molecular weight of one or more of the polymers may be altered such that inflation of the balloon 102 to deploy the stent 104 does not break a continuity of the coating 120 across the interconnected space regions 114. In other words, the coating 120 is configured to deform and/or extend over the entirety of the pre-crimped stent 104 as the balloon 102 is inflated and the pre-crimped stent 104 expands, such that the coating 120 continues to fully bridge the interconnected space regions 114. In one example embodiment, the PLLA-PLGA polymer matrix has a weight-average molecular weight in a range of about 800,000 g/mol to about 300,000 g/mol. In another embodiment, the weight-average molecular weight is between about 120,000 g/mol and about 250,000 g/mol, corresponding to molecular weight ranges used in bioabsorbable PLLA scaffold materials for cardiovascular and endovascular implants.

In some embodiments, a pre-crimped stent 104 mounted on a balloon 102 is coated by spray application of a coating solution 120 while the apparatus 100 is installed in a coating machine. The coating machine may include, without limitation, a spray nozzle unit, a protective enclosure or tube, a mandrel fixture, and/or a holder configured to support and rotate the implantable apparatus 100. The spray nozzle unit may be configured to dispense the coating solution 120, which may include one or more drugs and one or more biodegradable polymers dissolved in a low-boiling-point solvent. In some embodiments, the coating solution 120 is supplied to the spray nozzle unit from a feeding cup or reservoir associated with the coating machine.

By way of example, a pre-crimped stent 104 mounted on a balloon 102 and having dimensions of approximately 2.25 mm by 20 mm may be installed in a coating machine. A coating solution 120 comprising one or more drugs and one or more polymers may be prepared, and approximately 1 mL of the coating solution 120 may be applied to the pre-crimped stent 104-balloon 102 assembly by spray coating while the assembly is mounted in the coating machine. The spray coating may be performed under controlled conditions, including an inert gas pressure in a range of about 0.5 to about 4.0 psi and rotation of the coating machine or mounted assembly at a speed in a range of about 5 to about 40 revolutions per minute. Following application of the coating solution 120, the coated assembly may be allowed to dry at ambient temperature for a period of approximately five minutes to permit evaporation of residual solvent.

In some embodiments, the coating machine includes a rotatable mandrel configured to support the drug-delivering implantable apparatus 100 during coating. The pre-crimped stent 104 mounted on the balloon 102 may be secured to the rotatable mandrel and rotated during spray application such that the outer surface 117 of the assembly is uniformly exposed to a spray nozzle unit. In this manner, the coating 120 may be deposited on abluminal 108 and side surfaces 112 of the stent 104 and strut components 106a-106g, as well as on exposed portions 116 of the balloon 102 that extend radially and are exposed through the interconnected space regions 114 defined between adjacent strut components 106a-106g of the stent 104.

Referring now to FIGS. 4A, 4B and 5, when the coated pre-crimped stent 104 mounted on the balloon 102 is positioned within a body lumen 122, the balloon 102 may be expanded at a nominal inflation pressure, for example in a range of about 6 to about 9 atmospheres. Radial expansion of the balloon 102 may correspondingly expand the stent 104 and mechanically deform the coating 120 disposed on the outer surface 117 of the pre-crimped stent 104-balloon 102 assembly.

In the pre-crimped state, the stent 104 mounted on the balloon 102 may have an outer diameter 115 in a range of about 0.8 mm to about 1.4 mm, suitable for percutaneous delivery. Upon expansion of the balloon 102, the stent 104 may be configured to expand to a deployed diameter 119 substantially corresponding to the target vessel, for example in a range of about 2.0 mm to about 4.0 mm for coronary applications and up to about 6.0 mm or greater for peripheral vascular applications.

Expansion of the stent 104 from the pre-crimped diameter 115 to the deployed diameter 119 produces circumferential and longitudinal tensile forces on the coating 120 bridging the interconnected space regions 114, causing the coating 120 to stretch and reconfigure into the homogenous cylindrical film 202.

As a result of this balloon-driven deformation, the coating 120 may extend and reconfigure to form a continuous, flexible, homogenous cylindrical film 202 substantially circumscribing the stent 104 and contacting the vascular body wall 204 of the body lumen 122. The ability of the coating 120 to deform without cracking is facilitated by selection of the polymer matrix to have a molecular weight and mechanical properties suitable for tensile deformation during balloon 102 expansion.

For example, a stent having dimensions of approximately 2.25 mm by 20 mm may include a coating 120 having a drug concentration in a range of about 0.7 micrograms per square millimeter to about 1.8 micrograms per square millimeter, thereby providing enhanced circumferential drug delivery at a target site within the body lumen 122.

In some embodiments, the pre-crimped stent 104 assembly mounted on the balloon 102 includes a homogenous coating 120 including one or more drugs and an associated polymeric matrix selected to provide sufficient elasticity and plasticity to permit deformation without cracking during balloon 102 expansion. In some embodiments, the elasticity and/or plasticity of the polymeric matrix is adjusted by selecting an appropriate molecular weight, for example a molecular weight comparable to bioabsorbable scaffold materials used in cardiovascular applications. The homogenous coating 120 may be applied to the external surfaces of the stent 104, including abluminal 108 and side surfaces 112 of the strut components 106a-106g, and to portions 116 of the balloon 102 exposed through the stent 104, including regions extending beyond the first and second ends 109, 111 of the stent 104 by approximately 0.5 mm to 1.0 mm, thereby providing substantially complete coverage of the exterior of the drug delivery apparatus 100 assembly. Upon inflation of the balloon 102, the coating 120 mechanically deforms and extends such that the coating 120 is present across the strut components 106a-106g and bridges the interconnected space regions 114 defined between adjacent strut components 106a-106g.

In one embodiment, the coating 120 on a pre-crimped stent 104 mounted on the balloon 102 includes sirolimus dispersed within a biodegradable polymer matrix selected from a poly-L-lactide (PLLA) family of copolymers. In this embodiment, the stent 104 assembly includes a cobalt chromium stent 104 that is coated in the pre-crimped state. The coating 104 is disposed on the abluminal surfaces 108 and side surfaces 112 of the plurality of strut components 106a-106g of the stent 104, as well as on portions 116 of the balloon 102 that extend radially and are exposed through the interconnected space regions 114 defined between adjacent strut components 106a-106g. Following deployment of the drug delivery apparatus 100 within a body lumen, the biodegradable poly-L-lactide matrix undergoes hydrolytic degradation to form lactic acid, which is subsequently metabolized within the body to carbon dioxide and water over a period of approximately six to eight months.

In some embodiments, the coating 120 applied to the pre-crimped stent 104 mounted on the balloon 102, including sirolimus dispersed within a biodegradable polymer matrix selected from a poly-L-lactide family of copolymers, is prepared from a coating solution 120. In one example embodiment, sirolimus is dissolved in a solvent, such as methanol, at a concentration in a range of about 10 μg/mL to about 200 μg/mL, for example approximately 50 μg/mL, to form a drug solution. After complete dissolution of the sirolimus, a polymer selected from a biodegradable poly-L-lactide family of copolymers is added to the drug solution at a concentration in a range of about 1 mg/mL to about 20 mg/mL, for example approximately 5 mg/mL, to form a polymer-drug mixture. The resulting coating solution 120 is subsequently degassed, for example by ultrasonic treatment for a period of about 1 to 5 minutes, to remove entrapped gases prior to application to the pre-crimped stent 104 mounted on the balloon 102.

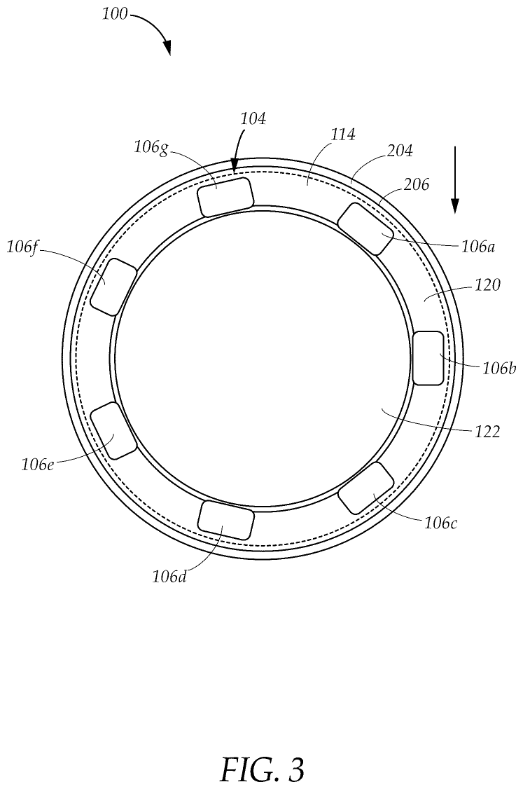

FIG. 1 illustrates an example embodiment of a coated pre-crimped stent 104 mounted on a balloon 102 in a representative coating configuration. FIG. 2 illustrates a cross-section of the coating 120 after radial expansion of the balloon 102, showing formation of a continuous, homogenous cylindrical film 202. FIG. 3 illustrates a cross-section of the coating 120 formation after deployment of the implantable apparatus 100 within a coronary vasculature.

In one exemplary embodiment of the present disclosure, a homogenous coating 120 including one or more drugs and an associated biodegradable polymeric matrix is disposed on outer surfaces 117 of a stent 104 while the stent 104 is in a pre-crimped state on a balloon 102. When the drug delivery implantable apparatus 100 is deployed within a coronary or peripheral vascular lumen 122, the balloon 102 is expanded at the target site for a controlled inflation period, for example in a range of about 45 seconds to about 60 seconds. During this inflation period, radial expansion of the balloon 102 may mechanically deform the coating 120 under wet physiological conditions, causing the coating 120 to extend and reconfigure into a continuous, homogenous cylindrical film 202. Formation of the cylindrical film 202 may be enabled by selection of the polymer matrix to have a molecular weight and mechanical properties sufficient to permit tensile deformation without cracking during balloon 102 expansion. As illustrated in FIG. 3, the cylindrical film 202 may be circumferentially apposed to the inner wall 204 of the arterial lumen 122, thereby delivering drug uniformly across the lesion and providing enhanced area-based drug coverage.

Various embodiments of the disclosed implantable drug delivery apparatus 100 and system 200 are configured to address lesions within a body lumen 122 that are associated with one or more medical conditions, including restenosis, occlusion of a body lumen 122, atherosclerosis, myocardial infarction, and plaque accumulation. The body lumen 122 may include, for example, a blood vessel, urethra, esophagus, ureter, or bile duct. In one embodiment, the coated pre-crimped stent 104 mounted on a balloon 102 is configured for use in coronary or peripheral arteries, including in patients exhibiting diabetic vascular disease.

In some embodiments, the coating 120 is selectively applied to the outer surface 117 of the pre-crimped stent 104 mounted on the balloon 102 such that the luminal surface 110 of the stent 104 assembly lacks coating. Correspondingly, balloon regions 118 positioned beneath the luminal surfaces 110 of the strut components 106a-106g also remain free of the coating 120. In some embodiments, this configuration limits drug exposure to circulating blood and is intended to support re-endothelialization following deployment of the apparatus 100 or system 200, particularly in coronary and peripheral vascular applications, where exposure of the luminal stent surface to blood flow is desirable.

Upon deployment within a coronary or peripheral artery, the homogenous cylindrical film 202 formed by balloon 102-driven deformation of the coating 120 may be circumferentially retained within the lumen 122 and apposed to the vessel wall 204. The circumferential configuration of the bioabsorbable cylindrical film 202 may enable both an initial burst release and a sustained release of the therapeutic agent over an extended period, ranging from days to months. This configuration may be particularly advantageous for treating restenosis or recurrent stenosis and for inhibiting uncontrolled cellular proliferation within the lumen 122, including in diabetic patients.

In one example embodiment, the coated pre-crimped stent 104 mounted on the balloon 102 includes a stent 104 having dimensions of approximately 3.00 mm by 20 mm. The coating 120 composition may include sirolimus in an amount of approximately 5 mg incorporated into a coating solution 120. In this example, the sirolimus is dissolved in an organic solvent, such as methanol, within a measuring vessel, followed by preparation of a polymer-drug solution having a theoretical concentration of approximately 50 μg/mL after degassing. A corresponding control or standard solution may be prepared for comparative analysis.

Drug content associated with the coated stent 104 may be characterized using analytical techniques such as high-performance liquid chromatography (HPLC). In one example, samples derived from the coated pre-crimped stent mounted on the balloon are filtered and introduced into an HPLC system equipped with a UV-VIS detector and a C18 column. Representative operating parameters may include a flow rate of approximately 1 mL/min, a detection wavelength of about 277 nm, an injection volume of approximately 20 μL, a column temperature of about 40° C. (+2° C.), and a run time of approximately 12 minutes. Such analysis confirms drug loading and supports characterization of drug distribution associated with the coated implantable device.

FIG. 4A illustrates an example embodiment of an implantable drug delivery system 200 positioned within a vascular body lumen 122 defined by a vascular body wall 204. The system 200 includes a balloon catheter 103 including an expandable balloon 102 and a pre-crimped stent 104 mounted on the balloon 102. The stent 104 includes a plurality of interconnected strut components 106 arranged in a mesh-like configuration defining a plurality of interconnected space regions 114. Each strut component 106 includes an abluminal surface 108, a luminal surface 110, and one or more side surfaces 112.

In the configuration shown in FIG. 4A, the balloon 102 is in a non-expanded state, and a coating 120 is disposed on the abluminal surfaces 108 and side surfaces 112 of the strut components 106 and on exposed portions 116 of the balloon 102 that extend radially through the interconnected space regions 114. Adjacent sections 118 of the balloon 102 disposed beneath the luminal surfaces 110 of the strut components 106 remain devoid of coating 120. The coating 120 bridges across the interconnected space regions 114 while the stent 104 is in the pre-crimped state, thereby forming a continuous coating 120 structure spanning between adjacent strut components 106.

In some embodiments, the coating 120 further extends longitudinally beyond the length 113 of the stent 104 onto corresponding proximal and distal ends 107, 105 of the balloon 102, by a distance between about 0.5 mm and about 1.0 mm. This configuration promotes formation of a continuous cylindrical film upon expansion, including at transition regions between the stent and the balloon.

In some embodiments, the stent 104 is formed from cobalt chromium, stainless steel, nitinol, platinum-chromium alloys, and/or other metallic or composite materials commonly used in vascular stents. The balloon 102 may be formed from compliant, semi-compliant, or non-compliant polymeric materials, including polyethylene terephthalate (PET), nylon, Pebax®, polyurethane, or multilayer composite balloon structures.

FIG. 4B illustrates the implantable drug delivery system 200 of FIG. 4A after radial expansion of the balloon 102 within the vascular body lumen 204. Expansion of the balloon 102 causes corresponding radial expansion of the stent 104 and mechanical deformation of the coating 120. As shown in FIG. 5, while still referring to FIGS. 4A and 4B, the coating 120 may undergo tensile stretching across the interconnected space regions 114, thereby extending between adjacent strut components 106.

During balloon 102 expansion, the coating 120 bridging adjacent strut components 106a-106g may experience tensile strain resulting from increasing separation between the strut components 106a-106g and expansion of the interconnected space regions 114. The coating 120 is selected and applied such that it undergoes tensile stretching across the interconnected space regions 114 without tearing, cracking, or delaminating.

In some embodiments, the tensile strain experienced by the coating 120 during balloon 102 expansion may be at least 30%, and in certain embodiments 50% to 150%, based on the change in projected area of the interconnected space regions 114 between the pre-crimped and expanded states.

As a result of this balloon-driven deformation, the coating 116 may reconfigure into a continuous, homogenous cylindrical film 202 that circumscribes the abluminal surfaces 108 of the stent 104 and circumferentially contacts the inner surface 206 of the vascular body wall 204. In some embodiments, the homogenous cylindrical film 202 is devoid of apertures corresponding to the interconnected space regions 114 of the stent 104, thereby providing continuous circumferential coverage and treatment of the entire vascular body wall 204.

In some embodiments, the coating 120 extends longitudinally beyond the distal end 105 and proximal end 107 of the stent 104 and onto corresponding distal and proximal ends 105, 107 of the balloon 102 by a distance in a range of about 0.5 mm to about 1.0 mm. Although the balloon catheter 103 shaft may extend through the balloon 102 at the distal and proximal ends 105, 107, the coating 120 may conform to the external balloon 102 surface and remain continuous around the circumference of the balloon 102 in these regions.

Upon expansion of the balloon 102, the coating 120 disposed on the distal and proximal ends 105, 107 of the balloon 102 may cooperate with the coating 120 disposed on the stent 104 to form a continuous homogenous cylindrical film 202 that spans the full treatment length, including transition regions adjacent the first and second ends 109, 111 of the stent 104. This configuration may prevent formation of untreated gaps at the ends 109, 111 of the stent 104 and/or promote complete circumferential coating of the vascular body wall 204 across the entire treatment area.

In some embodiments, the deformation behavior illustrated in FIG. 4B is enabled by selection of a polymer matrix having an elasticity and plasticity sufficient to deform without cracking during balloon 102 expansion. In some embodiments, the polymer matrix includes one or more polymers from a poly-L-lactide family, including PLLA or PLLA-PLGA copolymers, selected to have a molecular weight sufficient to permit tensile deformation in response to balloon 102 expansion. Alternative polymer matrices may include other biodegradable polymers such as polycaprolactone, poly(trimethylene carbonate), or blends thereof, provided that the resulting coating 120 exhibits comparable mechanical deformation behavior.

In certain embodiments, the homogenous cylindrical film 202 remains continuously apposed to the vascular body wall 204 upon deflation of the balloon 102, such that drug delivery continues after balloon 102 withdrawal.

Referring now to FIG. 5, another embodiment of the implantable drug delivery system 200 is shown following deployment, highlighting the homogenous cylindrical film 120 formed along the length 113 of the expanded stent 104 and extending to and substantially circumscribing the proximal and distal ends 105, 107 of the balloon 102. In this embodiment, the cylindrical film 202 is shown in circumferential contact with the vascular body wall 204 along substantially the entire treated length of the vascular body lumen 122.

In some embodiments, the coating 120 includes a polymer matrix containing one or more drugs, such as anti-restenotic, anti-proliferative, anti-inflammatory, antithrombotic, immunosuppressive, and/or cytostatic agents. In some embodiments, the drug includes sirolimus or a limus-family analog dispersed within a biodegradable polymer matrix. In certain embodiments, the polymer matrix may be configured to degrade over time by hydrolysis into biologically metabolizable byproducts, such as lactic acid, which may subsequently be metabolized to carbon dioxide and water.

In some embodiments, the circumferential configuration of the homogenous cylindrical film 202 facilitates uniform area-based drug delivery across the vascular body wall 204, as opposed to drug delivery limited to discrete strut contact regions. In addition, the homogenous continuous film 202 structure may provide a moderate tensile barrier that may inhibit migration of thrombotic debris through the stent 104 structure while permitting physiological vessel healing.

Although FIGS. 4A, 4B, and 5 illustrate balloon-expandable embodiments, it will be understood that the disclosed coating architecture and deformation behavior may be applied to alternative stent 104 geometries and balloon 102 configurations, including tapered balloons, stepped balloons, or balloons having variable compliance along their length, without departing from the scope of the invention.

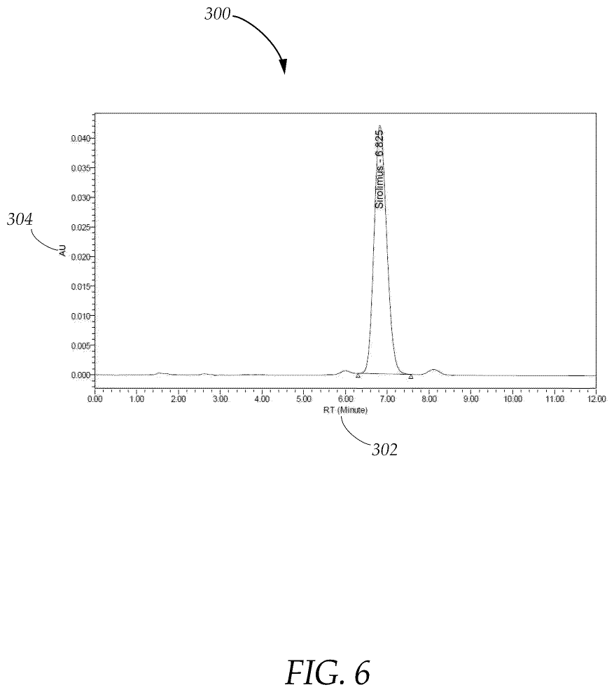

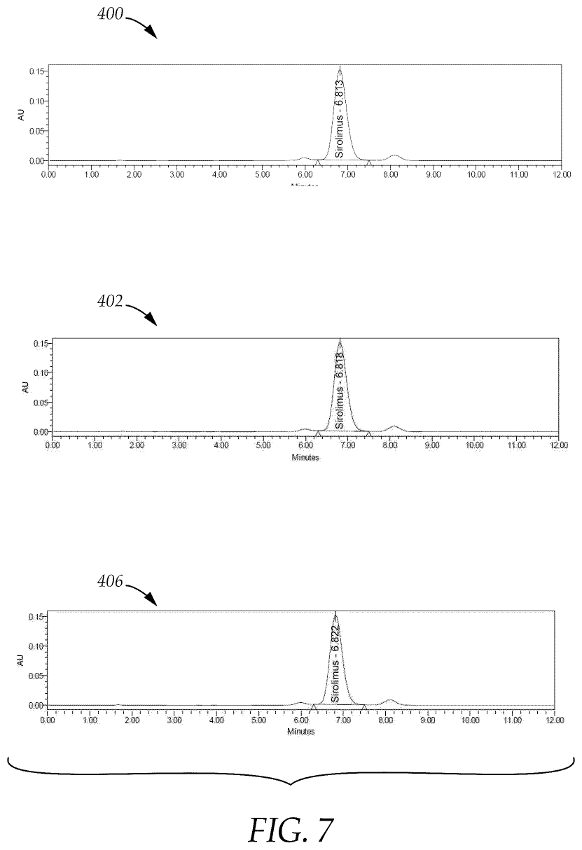

Referring now to FIGS. 6 and 7, while also referring to FIG. 1, FIG. 6 illustrates a representative chromatogram 300 obtained from high-performance liquid chromatography (HPLC) analysis over time 302 of a sample derived from a coated pre-crimped stent 104 mounted on a balloon 102. As shown in FIG. 6, the average chromatographic peak area corresponding to the drug present 304 in the sample is approximately 878,217.

FIG. 7 illustrates representative chromatograms 400, 402, 406 obtained from HPLC analysis of a control or standard solution. As shown, the average chromatographic peak area corresponding to the drug in the control solution is approximately 3,147,981.

The amount of drug associated with the coated pre-crimped stent mounted on the balloon may be calculated by comparing the sample chromatographic peak area to the control chromatographic peak area using the following relationship:

$$Drug content = ( \frac{Sample area}{Standard area} ) \times ( \frac{Standard weight}{Dilution} ) \times ( \frac{Dilution}{Sample} ) \times Potency$$

Using the representative average peak area values shown in FIGS. 6 and 7, the calculated drug content associated with a coated pre-crimped stent 104 having dimensions of approximately 3.00 mm by 20 mm is approximately 136.56 μg.

In some embodiments, the homogenously coated implantable apparatus 100 having an enhanced circumferential drug delivery area is configured for use in clinical scenarios associated with acute myocardial infarction (AMI) and thrombus-containing lesions (TCL). In such conditions, conventional treatment approaches often include thrombolytic agents or thrombus aspiration followed by implantation of a drug-eluting stent. While these approaches may remove thrombotic material, they can be associated with slow-flow or no-flow phenomena caused by residual debris, acute thrombus formation, or distal embolization.

Referring again to FIGS. 1 and 5, in some embodiments, the implantable drug delivery apparatus 100 provides a homogenous cylindrical film 202 having a moderate tensile strength formed by balloon 102-driven deformation of the coating 120. The circumferential film configuration is capable of limiting migration of thrombotic debris into the bloodstream while maintaining continuous contact with the vascular body wall 204. As a result, the apparatus 100 and system 200 may be configured to reduce the likelihood of acute, sub-acute, and late thrombus formation within the lumen and to mitigate slow-flow or no-flow conditions associated with conventional stent deployment.

Various embodiments of the present disclosure thus provide a circumferential, bioabsorbable film 202 that may be formed from a drug-containing polymeric matrix having enhanced elasticity and plasticity. The film 202 may be configured to deliver therapeutic agents over an expanded surface area of the vascular body wall 204 and combine features traditionally associated with drug-eluting stents 104 and drug-eluting balloons 102. By increasing the availability and uniformity of drug delivery within the body lumen 122, the disclosed apparatus 100 and system 200 may support inhibition of restenosis and uncontrolled cellular proliferation while maintaining favorable hemodynamic conditions.

The coating 120 described herein is not configured to increase adhesive retention between the stent 104 and the balloon 102 during delivery. Rather, retention of the stent 104 on the balloon 102 prior to deployment is achieved by crimping. The coating 120 is instead configured to deform mechanically during balloon 102 expansion to form a circumferential film 202 contacting the vascular body wall 204 after deployment.

Those skilled in the art will realize that the above-recognized advantages and other advantages described herein are merely exemplary and are not meant to be a complete rendering of all of the advantages of the various embodiments of the disclosure.

In the foregoing provisional specification, specific embodiments of the disclosure have been described. However, one of ordinary skill in the art appreciates that various modifications and changes can be made to the disclosure without deviating from the scope of the disclosure. Accordingly, the provisional specification is to be regarded in an illustrative rather than a restrictive sense, and all such modifications are intended to be included within the scope of the disclosure.

We claim:

1. An apparatus for delivering a drug to a vascular body lumen, comprising:
a pre-crimped stent mounted on a balloon and configured to be disposed within a vascular body lumen defined by a vascular body wall, wherein the pre-crimped stent comprises a mesh-like configuration having a plurality of interconnected space regions defined by a plurality of strut components, wherein each of the plurality of strut components comprises an abluminal surface, a luminal surface, and a side surface, and wherein the balloon comprises a plurality of exposed portions exposed through the interconnected space regions and a plurality of adjacent sections beneath the luminal surface; and
a coating contacting the abluminal surface and the side surface of each of the plurality of strut components and the exposed portions of the balloon, wherein the adjacent sections of the balloon lack coating, wherein the coating bridges across the interconnected space regions of the pre-crimped stent, wherein the coating is configured to deform and extend in response to expansion of the balloon to form a continuous, homogenous cylindrical film circumscribing the abluminal surface, wherein formation of the homogenous cylindrical film is driven by expansion of the balloon, wherein the homogenous cylindrical film comprises a polymer matrix comprising one or more drugs and one or more polymers from a poly-L-lactide family, and wherein, upon expansion of the balloon within the vascular body lumen, the homogenous cylindrical film covers a continuous, circumferential area of the vascular body wall.

2. The apparatus of claim 1, wherein the coating further contacts a proximal end and a distal end of the balloon such that the homogenous cylindrical film further substantially circumscribes the proximal end and the distal end.

3. The apparatus of claim 1, wherein deformation of the coating in response to expansion of the balloon comprises tensile stretching of the coating across the interconnected space regions.

4. The apparatus of claim 1, wherein, upon expansion of the balloon within the vascular body lumen, the homogenous cylindrical film contacts a circumferential inner surface of the vascular body lumen.

5. The apparatus of claim 4, wherein the homogenous cylindrical film continuously coats the circumferential inner surface.

6. The apparatus of claim 5, wherein the homogenous cylindrical film remains continuously in contact with the circumferential inner surface upon deflation of the balloon.

7. The apparatus of claim 1, wherein the coating comprises a polymer having an elasticity and a plasticity sufficient to deform without cracking in response to expansion of the balloon.

8. The apparatus of claim 7, wherein the polymer comprises a molecular weight sufficient to permit tensile deformation in response to expansion of the balloon.

9. The apparatus of claim 2, wherein the coating extends longitudinally onto the distal end and the proximal end of the balloon by a distance between 0.5 mm and 1.0 mm beyond a length of the pre-crimped stent.

10. The apparatus of claim 1, wherein the luminal surface of each of the plurality of strut components and the adjacent sections of the balloon beneath the luminal surface remain devoid of the coating after formation of the homogenous cylindrical film.

11. A system for delivering a drug to a vascular body lumen, comprising:
a balloon catheter comprising an expandable balloon, wherein the balloon catheter is configured to be disposed within a vascular body lumen defined by a vascular body wall;
a pre-crimped stent mounted on the balloon, the pre-crimped stent comprising a plurality of strut components defining a plurality of interconnected space regions; and
a coating disposed on an outer surface of the pre-crimped stent and on exposed portions of the balloon exposed through the interconnected space regions, wherein the coating bridges across the interconnected space regions of the pre-crimped stent, wherein the coating is configured to deform and extend in response to expansion of the balloon to form a continuous, homogenous cylindrical film circumscribing the pre-crimped stent, wherein formation of the homogenous cylindrical film is driven by expansion of the balloon, and wherein the homogenous cylindrical film circumferentially contacts a surface of the vascular body wall upon expansion of the balloon.

12. The system of claim 11, wherein the pre-crimped stent comprises a plurality of strut components, each of the plurality of strut components comprising an abluminal surface, a luminal surface, and a side surface, and wherein the outer surface of the pre-crimped stent corresponds to the abluminal surface and the side surface of each of the plurality of strut components.

13. The system of claim 12, wherein balloon regions disposed beneath the luminal surface of each of the plurality of strut components remain devoid of the coating.

14. The system of claim 11, wherein the deformation of the coating in response to expansion of the balloon comprises tensile stretching of the coating across the interconnected space regions.

15. The system of claim 11, wherein the homogenous cylindrical film is devoid of apertures corresponding to the interconnected space regions of the pre-crimped stent.

16. The system of claim 11, wherein the coating comprises a polymer selected to have an elasticity and a plasticity sufficient to deform without cracking in response to expansion of the balloon.

17. The system of claim 16, wherein the polymer comprises a molecular weight selected to permit tensile deformation in response to expansion of the balloon.

18. The system of claim 11, wherein the coating extends longitudinally onto the distal end and the proximal end of the balloon by a distance between 0.5 mm and 1.0 mm beyond a length of the pre-crimped stent.

19. The system of claim 11, wherein the coating comprises a polymer matrix containing one or more drugs selected from the group consisting of anti-restenotic, anti-proliferative, anti-inflammatory, antithrombotic, immunosuppressive, and cytostatic agents.

20. The system of claim 11, wherein the homogenous cylindrical film is formed in vivo in response to the coating coming into contact with fluid within the vascular body lumen.
